(19) United States
(12) Patent Application Publication
(10) Pub. No.: US 2023/0158181 A1
(43) Pub. Date: May 25, 2023

(54) A Steam Disinfestation Inline System and Apparatus for Agricultural Bud or Leaf Products

(71) Applicant: The State of Israel, Ministry of Agriculture & Rural Development, Agricultural Research Organization, Rishon Lezion (IL)

(72) Inventors: Rafi REGEV, Rishon Lezion (IL); Ze'ev SCHMILOVITCH, Yehud (IL); Ahron HOFFMAN, Givataim (IL); Dani ESHEL, Pardes Hanna-Karkur (IL); Dekel MEIR, Midreshet Ben-Gurion (IL)

(21) Appl. No.: 17/440,806

(22) PCT Filed: Mar. 18, 2020
(86) PCT No.: PCT/IB2020/052470

§ 371 Date: Sep. 20, 2021

Related U.S. Application Data
(60) Provisional application No. 62/820,883, filed on Mar. 20, 2019.

(51) Int. Cl. A61L 2/00; A61L 2/26; B65G 15/30

(52) U.S. Cl. CPC A61L 2/0023; A61L 2/26; B65G 15/30; A61L 2202/122; A61L 2202/123; A61L 2202/14; A61L 2202/15; B65G 2201/0202

(57) ABSTRACT

The present invention provides systems and methods for treating agricultural particulates, such as buds, Cannabis buds, herbs, leaves, and the like, with a treatment agent, such as steam, as the agricultural products move forward along a pathway created by a conveyer, in a chamber, enclosed by an aligned ceiling and floor, and oppositely disposed lateral walls. The exposure times to the steam, created by the intervals and lengths in which steam enters the chamber results in the elimination (destruction) and/or neutralization of the pathogens on the agricultural particulates, while the agricultural particulates remain undamaged and active.

### CROSS-REFERENCES TO RELATED APPLICATIONS

This application is related to and claims priority from commonly owned U.S. Provisional Patent Application Ser. No. 62/820,883, entitled: A Steam Disinfection Inline System and Apparatus for Agricultural bud or leaf Products, filed on Mar. 20, 2019, the disclosure of which is incorporated by reference in its entirety herein.

### TECHNICAL FIELD

The present invention relates to the disinfestation of agricultural and other products in their bud or leaf form.

### BACKGROUND OF THE INVENTION

There is an increasing public concern over the level of pesticide residues in agricultural products. Many previous fungicides have become ineffective due to the development of pathogens resistant to these fungicides. As a result, the use of pesticides and fungicides for post-harvest control of diseases in agricultural products has been substantially restricted and considerable research and development has been conducted for using other post-harvest treatments of agricultural products to extend their storage life.

Heat treatment for disinfesting agricultural products leaves no pesticide residues and is considered as both environmental friendly and human friendly. However, when heat is applied to the complete outer surface of the agricultural products sufficient for post-harvest disease control, there is a danger that hot spots may be produced on the outer surface of the agricultural products, damaging them, either partially or completely. Such damaged products become vulnerable to necrotrophic pathogens.

### SUMMARY OF THE INVENTION

The present invention provides systems and methods for treating agricultural particulates, such as buds, Cannabis buds, herbs, leaves, and the like, with a treatment agent, such as steam, as the agricultural products move forward along a pathway created by a conveyer, in a chamber, enclosed by an aligned ceiling and floor, and oppositely disposed lateral walls. The exposure times to the steam, created by the intervals and lengths in which steam enters the chamber results in the elimination (destruction) and/or neutralization of the pathogens on the agricultural particulates, while the agricultural particulates remain undamaged and active.

Embodiments of the invention are directed to a disinfestation method comprising: placing a plurality of particulates along a pathway for causing movement of the particulates to expose the exterior surface of the particulates to treatment substances; providing a treatment chamber along the pathway; and, treating the particulates with a treatment substance while the particulates are passing through the treatment chamber.

Optionally, the treatment substance is steam.

Optionally, the treatment substance is a treatment agent.

Optionally, the rotational pathway is created by belt conveyer.

Optionally, the particulates include Cannabis buds.

Optionally, the particulates are in a bud or leaf form.

Optionally, the size of the particulates ranges approximately between 1 mm and 6.5 cm.

Optionally, the exposure period of the particulates to the treatment substance ranges approximately between 0-20 seconds.

Optionally, the method additionally comprising introducing the steam into the treatment chamber through at least one nozzle in the treatment chamber.

Optionally, the steam is introduced through the at least one nozzle at a temperature of approximately 120°-140° C.

Optionally, the steam causes the particulates surface temperature to reach a temperature of approximately 70° C.

Embodiments of the invention are directed to a system for treating agricultural particulates comprising: a chamber configured for receiving a treatment agent in the interior of the chamber; a conveyer extending at least partially through the chamber, the conveyer configured for maintaining a forward moving path for the agricultural particulates, the conveyer is divided into two sections, an upper section and bottom section, the agricultural particulates are transferred from the upper section to the bottom section, such that the agricultural particulates are turned over, exposing all the exterior of the particulates to the treatment agent.

Optionally, the system additionially comprising a steam source for providing steam as the treatment agent to the interior of the chamber.

Optionally, the system additonally comprising a computerized controller configured for controlling the stream source, such that steam is provided to the chamber at predetermined intervals.

Optionally, the computerized controller is configured for controlling the stream source, such that steam is provided to the chamber at the predetermined intervals for predetermined lengths of time.

Optionally, the chamber includes an opening for receiving the agricultural particulates at a proximal end of the chamber, and an opening through which the agricultural particulates are exiting from the chamber at a distal end.

Optionally, the forward moving path is from the opening at the proximal end of the chamber to the opening at the distal end of the chamber.

Optionally, the conveyer is driven by a motor.

Optionally, the motor is controlled by the computerized controller.

Optionally, the computerized controller is in communication with a thermal camera configured for monitoring the surface temperature of the agricultural particulates.

Unless otherwise defined herein, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the present invention are herein described, by way of example only, with reference to the accompanying drawings. With specific reference to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Attention is now directed to the drawings, where like reference numerals or characters indicate corresponding or like components.

In the drawings:

FIG. 1 is a side view of the system according to an embodiment of the present invention; and,

FIG. 2 is a side view of the system according to another embodiment of the present invention.

### DESCRIPTION OF THE PREFERRED EMBODIMENTS

The present invention is not limited in its application to the details of construction and the arrangement of the components set forth in the following description. The invention is capable of other embodiments, or of being practiced or carried out in various ways. Also, it is to be understood that the phraseology and terminology employed herein is for the purpose of description and should not be regarded as limiting.

The present invention provides a system for disinfesting agricultural products, as particulates, such as buds, Cannabis buds, herbs, leaves, and the like, from potential pathogens using short and efficient exposure to steam.

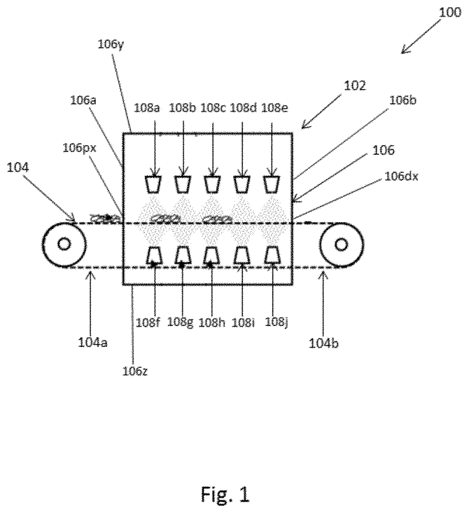

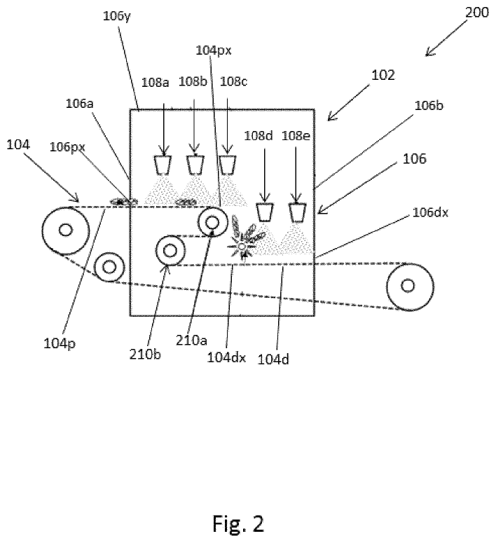

FIGS. 1 and 2 are side views of the systems 100 and 200 respectively for treating agricultural products, including agricultural particulates or particulates, such as buds, Cannabis buds, herbs, leaves, and the like hereinafter and collectively known as “agricultural particulates” or “particulates”, these terms used interchangeably herein.

FIG. 1 is a side view of the system 100. The system 100 includes a treatment unit 102 and a conveyer 104, e.g., a belt conveyer, on which the particulates are positioned, forming a pathway for the particulates, from a raw product to a treated product. The belt conveyer is of a mesh material allowing steam to contact particulates from both sides of the belt.

The system 100 is designed to accommodate, for example, particulates (and other materials) of between approximately 1 mm and 6.5 cm.

The treatment unit 102 includes a chamber or enclosure 106. A proximal portion 104a and a distal portion 104b of the conveyer 104 extend out of the chamber 106, so as to be driven by a motor (not shown) via gears or belts. The motor may be activated manually or controlled by a computerized controller (not shown), over wired and/or wireless networks, or combinations thereof. The conveyer 104 may include, for example, a screen mesh on which the particulates are positioned. The chamber 106 includes a proximal opening 106px in the side wall 106a of the chamber 106 and a distal opening 106dx in the parallel side wall 106b of the chamber 106. The raw particulates are entered into the chamber 106 via proximal opening 106px and are exited from the chamber 106 as treated particulates through proximal opening 106dx.

The chamber 106 includes one or more nozzles 108a-108j, for example, in the chamber upper wall 106y and lower wall 106z, through which steam enters the chamber 106, from a steam unit or source via pipes (not shown). The steam unit or source may be controlled by a computer, computerized controller or the like, as well as manually. The computer or manual control is such that steam, which is for example, water-based, is released from the steam unit at various times, including time intervals, for various time lengths.

The steam is at a temperature sufficient, such that all or substantially all, of the exterior (or surface) of the particulates is exposed to the steam in the chamber 106. The exposure to the steam eliminates and/or neutralizes pathogens for each of the particulates, for example, disinfesting each of the particulates without destroying or deactivating the particulates themselves.

The temperature of the steam entering from the steam unit or source may be controlled using a thermal camera (not shown) positioned within system 100. The thermal camera monitors the surface temperature of the treated particulates and adjusts the steam temperature accordingly so as to prevent heat damage to the particulates.

In addition to steam, other treatment substances or agents, either alone or in combination with steam, may also be transmitted into the chamber 106 for treatment of the particulates. The treatment agents, are typically gasses or liquids, but may also be solids, such as powders, for example, which are food safe, or UV light.

In operation, for example, the steam enters the chamber 106 through the steam nozzles 108a-108j, at temperatures of approximately 120° C.-140° C. This entry temperature is such that the steam contacts the particulates surface at temperatures sufficient to treat the particulates from pathogens and other harmful agents, but not cause heat damage to the particulates. The steam causes the surface temperature of the particulates to reach a temperature of approximately 70° C., as this temperature allows disinfesting of the particulates from pathogens and other harmful agents without causing heat damage to the particulates. The temperature of the steam declines due to the distance between the steam nozzles 108a-108j and the particulates on the conveyer 104. The exposure time of the particulates to the steam is, for example, restricted to a predetermined period of time, approximately 0-20 seconds, as provided manually or by a computer controller, and/or coupled with the exposure time to the steam, in order to prevent heat damage to the particulates.

FIG. 2 is a side view of the system 200 which is similar in construction and operation to apparatus 100, as detailed above, except where indicated. The system 200 includes one or more gears 210a-210b located within chamber 106. The Gears 210a-210b are positioned such that conveyer 104 is divided into two sections, an upper section 104p, and bottom section 104d. The end 104px of upper section 104p is positioned over a proximal portion 104dx of bottom section 104d. The transition of the particulates from the end 104px of upper section 104p to the proximal portion 104dx of bottom section 104d causes the particulate to flip over. The nozzles 108a-108e are positioned, for example, in the chamber upper wall 106y such that each nozzle is positioned at a similar distance from its corresponding conveyer region.

In operation, for example, the steam enters the chamber 106 through the steam nozzles 108a-108e. The raw particulates enter the chamber 106 through proximal opening 106px and are conveyed toward end 104px such that the steam contacts the particulates upper surface. At the end 104px, the particulates fall onto the proximal portion 104dx, such that the particulates are flipped and turned over, with their bottom surface facing the steam nozzles 108a-108e. The particulates are then conveyed from the proximal portion 104dx to the distal opening 106dx, while the steam contacts the particulates previous bottom surface.

### EXAMPLES

The following examples are not meant to limit the scope of the claims in any way. The following examples are put forth so as to provide those of ordinary skill in the art with a complete disclosure and description of how to make and use the described invention, and are not intended to limit the scope of the invention, nor are they intended to represent that the experiments below are all or the only experiments performed. Unless indicated otherwise, parts are parts by weight, molecular weight is weight average molecular weight, temperature is in degrees Centigrade, and pressure is at or near atmospheric.

### Example 1

### Disinfesting Cannabis Buds

Samples of Cannabis buds were exposed to stem for periods of 9 and 17 sec. The samples were disinfesting using the system disclosed in FIG. 2. The samples were assessed for contamination levels vs. treatment. The sanitation results are presented in table 1 below.

TABLE 1
Results of contamination levels vs. treatment

| | TIME | | TOTAL |
|---|---|---|---|
| SAMPLE | (SEC) | MOLD | COUNT |
| 1 | 0 | 3400 | 400000 |
| 2 | 9 | 0 | 16000 |
| 3 | 9 | 0 | 5400 |
| 4 | 9 | 0 | 0 |
| 5 | 9 | 0 | 0 |
| AVG 2-3 | 9 | 0 | 10700 |
| 6 | 17 | 0 | 1100 |
| 7 | 17 | 0 | 830 |
| 8 | 17 | 0 | 0 |
| 9 | 17 | 0 | 0 |
| AVG 6-7 | 17 | 0 | 965 |

The results show that the most efficient treatment was achieved in the 17 sec period.

While the invention has been described with respect to a limited number of embodiments, it will be appreciated that many variations, modifications and other applications of the invention may be made. Therefore, the claimed invention as recited in the claims that follow is not limited to the embodiments described herein.

1. A disinfestation method comprising:
placing a plurality of particulates along a pathway for causing movement of said particulates to expose the exterior surface of said particulates to treatment substances;
providing a treatment chamber along said pathway; and,
treating said particulates with a treatment substance while said particulates are passing through said treatment chamber.

2. The method of claim 1, wherein said treatment substance is steam.

3. The method of claim 1, wherein said treatment substance is a treatment agent.

4. The method of claim 1, wherein said rotational pathway is created by belt conveyer.

5. The method of claim 1, wherein said particulates include Cannabis buds.

6. The method of claim 1, wherein said particulates are in a bud or leaf form.

7. The method of claim 1, wherein the size of said particulates ranges approximately between 1 mm and 6.5 cm.

8. The method of claim 1, wherein the exposure period of said particulates to said treatment substance ranges approximately between 0-20 seconds.

9. The method of claim 2, additionally comprising introducing said steam into said treatment chamber through at least one nozzle in the treatment chamber.

10. The method of claim 8, wherein said steam is introduced through said at least one nozzle at a temperature of approximately 120°-440° C.

11. The method of claim 8, wherein said steam causes the particulates surface temperature to reach a temperature of approximately 70° C.

12. A system for treating agricultural particulates comprising:
a chamber configured for receiving a treatment agent in the interior of the chamber;
a conveyer extending at least partially through the chamber, the conveyer configured for maintaining a forward moving path for the agricultural particulates, the conveyer is divided into two sections, an upper section and bottom section, the agricultural particulates are transferred from the upper section to the bottom section, such that the agricultural particulates are turned over, exposing all the exterior of the particulates to the treatment agent.

13. The system of claim 12, additionially comprising a steam source for providing steam as the treatment agent to the interior of the chamber.

14. The system of claim 13, additonally comprising a computerized controller configured for controlling the stream source, such that steam is provided to the chamber at predetermined intervals.

15. The system of claim 14, wherein the computerized controller is configured for controlling the stream source, such that steam is provided to the chamber at the predetermined intervals for predetermined lengths of time.

16. The system of claim 12, wherein the chamber includes an opening for receiving the agricultural particulates at a proximal end of the chamber, and an opening through which the agricultural particulates are exiting from the chamber at a distal end.

17. The system of claim 16, wherein the forward moving path is from the opening at the proximal end of the chamber to the opening at the distal end of the chamber.

18. The system of claim 12, wherein the conveyer is driven by a motor.

19. The system of claim 18, wherein the motor is controlled by the computerized controller.

20. The system of claim 14, wherein the computerized controller is in communication with a thermal camera configured for monitoring the surface temperature of said agricultural particulates.
